(19) United States
(12) Patent Application Publication
(10) Pub. No.: US 2007/0160553 A1
(43) Pub. Date: Jul. 12, 2007

(54) Use of agents for the cosmetic treatment of keratin-containing material

(75) Inventors: Thomas Kripp, Fraeikisch-Crumbach (DE); Beate Grasser, Hattersheim (DE); Anette Sallwey, Dreicid (DE)

Correspondence Address: Striker Striker and Stenby, 103 East Neck Road, Huntington, NY, 11743, US

(73) Assignee: WELLA AG, Darmstadt (DE)

(21) Appl. No.: 10/587,278

(22) PCT Filed: Jan. 12, 2005
(86) PCT No.: PCT/EP05/01442

§ 371 Date: Jul. 25, 2006

(30) Foreign Application Priority Data
Mar. 20, 2004 (DE) 10 2004 013 798.6

(51) Int. Cl. A61K 8/49; A61K 31/555

(52) U.S. Cl. 424/70.7; 514/355; 514/184

(57) ABSTRACT

The object of the present invention is the use of a compound of formula 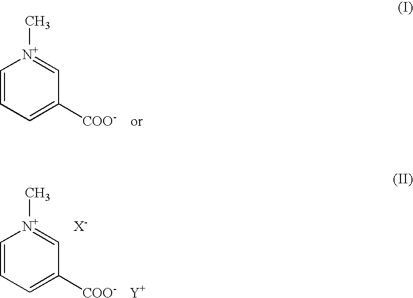 and of the salts thereof in an agent for repairing, strengthening and restructuring keratin-containing material and protecting such material from mechanical and chemical damage.

The present invention has for an object the use of an agent containing a compound of formula (I) 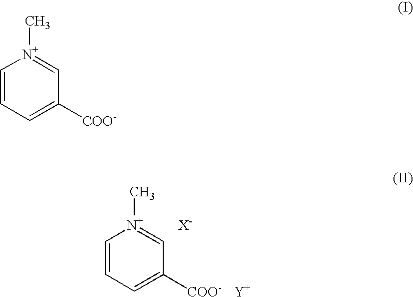
for repairing, strengthening and restructuring keratin-containing material, particularly keratin-containing skin appendages and preferably keratin fibers such as human hair, and protecting such material from mechanical and chemical damage.

It is known that keratin-containing material, particularly keratin fibers, is damaged by environmental factors (for example, energy-rich radiation), by the physiological status (for example by the age or health of the individual involved) or by mechanical and chemical effects. Such damage results in a dete-rioration of the mechanical properties of the affected material. Damage to the inner structure of keratin fibers manifests itself, for example, in a loss of hardness, firmness, breaking strength, tear strength or bundle tensile strength.

Human hair is usually exposed to a multiplicity of damaging influences, such as sunlight, heat, chlorinated water, sea water, mechanical stress owing to brushing, wearing a hairpiece, rubbing, washing and possibly to permanent wave treatments and to the use of cosmetic agents for bleaching or dyeing. Mechanical stresses cause damage primarily to the cuticula whereas heat, radiation, alkaline, reductive and oxidative treatments, on the other hand, attack directly the chemical structure of hair and cause a degree of damage that depends on the disposition, kind of effect, concentration and exposure time of the hair. It is particularly deleterious that hair with an increasing degree of previous damage reacts more sensitively to any other weakening effect. For example, undamaged hair tolerates well correctly applied permanent wave treatment. Repeated bleaching, however, causes considerable previous damage to the hair, so that the permanent wave treatment may lead to clearly evident damage and even cause individual hairs to break off.

On keratin fibers in particular, and especially on human hair, such effects manifest themselves particularly in reduced tear strength and poor combability. They are brought about by aging processes, caused primarily physiologically, or by physical (weathering), mechanical (combing, brushing) and chemical (dyeing, deforming) effects. On long hair, these effects are seen particularly in the hair tips. Chemical effects include primarily bleaching, oxidative dyeing and permanent wave treatment of hair, for which aggressive oxidants and reducing agents are used preferably in a strongly alkaline medium where they exert their full effect. Other chemical influences, for example water enriched in chlorine or salts, however, also exert a damaging effect on keratin-containing material.

It is neither possible nor desirable to protect hair consistently especially from the said effects. Moreover, according to the current state of the art there exists no method for hair deformation or color change that is effective without leaving a certain degree of damage at the site of the treatment. Because, after being created, hair is not subjected to any biological regeneration process, it is exclusively exposed to external influences. It would therefore be desirable to have an effective possibility to suppress undesirable side effects of certain cosmetic treatments of hair while retaining unlimited product effectiveness.

Commercial rinses and treatments contain as active substances mainly cationic surfactants or polymers, waxes and/or oils. The more damaged the hair, the more anionic groups are present at the surface. Cationic compounds are electrostatically attracted to this oppositely charged surface, whereas oils and waxes interact with the hydrophobic groups of keratin. For this reason, an improvement in the internal structure of hair cannot be attained with these hair-care products.

The underlying goal of the present invention was to provide an agent, particularly a cosmetic hair-treatment agent, intended for use in improving the condition of hair and which would eliminate the aforesaid drawbacks.

According to the invention, this objective is reached by use of a compound of formula (I) and/or (11) 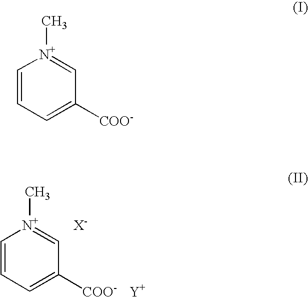
including the salts thereof, wherein X⁻ and Y⁻ stand for any inorganic or organic, monovalent or polyvalent physiologically unobjectionable anion or cation, in an agent for repairing, strengthening, and restructuring keratin-containing material and protecting it from mechanical and chemical damage.

X⁻ as the counterion can denote any conceivable organic acid group (base). In particular, X⁻ can be selected from the group consisting of formate, tartrate, oxalate, aspartate, glutamate, acetate, citrate or of inorganic groups such as chloride, bromide, iodide, sulfate, hydrogen sulfate, phosphate, monohydrogen or dihydrogen phosphate, hydroxide, carbonate and nitrate.

In a preferred embodiment of the invention, X⁻ stands for hydrochloride, acetate or citrate.

Y⁺ can be selected from the group of cations consisting of protons, alkali metals, preferably lithium, sodium and potassium, alkaline earth metals, preferably calcium and magnesium, subgroup metals, preferably aluminum, iron, zinc, copper, manganese and silver, ammonium groups or primary, secondary, tertiary or quaternary amines, hydrazides or hydroxylammonium groups.

Other embodiments of the present invention are indicated in the subclaims.

The compounds of formulas (I) and (II) refer to the known alkaloid trigonelline (1-methylpyridinium-3-carboxylate) which has been described in the literature and is also known under the denominations of 3-carboxy-1-methylpyridinium hydroxide, nicotinic acid N-methylbetaine or coffearine. Hence, the compounds (I) and (II) of the present invention comprise the denominations 1-methylpyridinium-3-carboxylate, trigonelline, coffearine, 3-carboxy-1-methylpyridinium hydroxide and nicotinic acid N-methyl betaine in synonymous manner.

The compounds of formula (I) and (II) can be prepared by synthesis or obtained by known methods from various plant extracts, for example from coffee beans (Coffea arabica), or from carob seed (Trigonella foenum graecum). In the latter case, the use according to the invention lies in the fact that the compounds of formulas (I) and (II) are present as constituents of a natural plant extract.

Unroasted coffee beans (Coffea arabica) or carob seed (Trigonella foenum graecum) are preferred for obtaining a compound of formula (I) or (II) or the alkaloid trigonelline as the active ingredient from natural sources.

It is irrelevant for the action according to the invention whether the compounds of formulas (I) and (II) are used as a synthetically obtained pure substance, as a purified extract isolated from natural sources or as a crude extract. Critical is only the content of active substance or active ingredient. The active ingredient can be used in its pure, betainic form of formula (I) or as a salt of formula (II), as previously described.

Tests of the efficacy of compounds of formulas (I) and/or (II) have shown that despite their betainic structure they did not exhibit on the hair any of the conventional hair-care effects.

This is in stark contrast to the known hair-care actions of other representatives of this class of substances, for example of betaine (trimethylammonioacetate) or carnitine [3-hydroxy-4-(trimethylammonio)butyric acid]. Whereas in appropriate formulations the two last-mentioned compounds brought about a pronounced improvement in combability and also definitely improved the luster of the hair, the compounds of formulas (I) and (II) produced none of these effects. Other hair-care criteria such as an increase in hair volume or an improvement in the hair fullness or hair styling were also not observed.

Surprisingly, however, we have found that the use of compounds of formulas (I) and/or (II) in hair-cosmetic products brings about a marked improvement in hair resistance to mechanical and chemical (particularly oxidative and alkaline) attacks.

Thus, rubbing tests which provide a measure of resistance to mechanical weakening have shown a significant increase in the lifetime of hair treated with the compounds of formulas (I) and/or (II) (FIG. 1). Tests for determining the triboelectric properties have also shown that depending on the use concentration of compounds of formulas (I) and (II) the electrostatic chargability of hair was reduced. Moreover, we found that compounds of formulas (I) and (II) markedly reduced the mechanical abrasion loss of artificial hair dyes so that the durability of hair tints was markedly improved. This is particularly surprising considering that in no comparative test did the formulation containing as active ingredient a compound of formula (I) and (II) show a reduction in the mechanical coefficient of friction. As stated hereinabove, neither the combability nor the disentanglement of the hair could be significantly improved. The protective function therefore is independent of the typical hair-care criteria and consists only of attenuating the effects of hair-damaging factors.

Hence, it was quite surprising to find that by use of compounds of formulas (I) and/or (II) or of the salts thereof the structure of keratin-containing material, particularly of keratin fibers (hair) is modified to an extent such that said compounds can be used advantageously for repairing, strengthening and restructuring keratin-containing material and protecting it from mechanical and chemical damage.

Besides protecting from mechanical effects, the preparations containing a compound of formula (I) and/or (II) also protect thoroughly against chemical and particularly oxidative stresses. For example, the presence of 1-methylpyridinium-3-carboxylate in permanent wave products brought about a significant increase in tear strength compared to otherwise identically treated hair. Tear strength is viewed as a parameter for determining damage to hair; the higher the force needed to tear a hair, the better is the hair quality. In individual cases, this protective effect was so great that the forces needed to cause tearing were as high as or even slightly higher than those noted in reference tests without the permanent wave treatment. This suggests that the repairing mechanism clearly extends beyond a normal protective function.

The damage necessarily caused by an alkaline treatment, which manifests itself most obviously in a certain dullness, lack-luster nature of the hair was also clearly reduced by use of a product containing a compound of formula (I) and/or (II). Thus, the luster values measured after treatment with an alkaline tinting agent were in all cases clearly higher with products containing a compound of formula (I) and/or (II) than for hair that had been treated with a conventional product.

On undamaged or unstressed hair, the active ingredient in the form of a compound of formula (I) and/or (II) shows none of the criteria of conventional hair care (improvement in combability, volume, feel or hair luster). Rather, said active ingredient exerts a marked protective function against the aforesaid factors and thus can attenuate, prevent or even partly eliminate the negative effects of aggressive treatment methods. In permanent wave preparations, in particular, said active ingredient exerts a repairing action. Compounds of formulas (I) and/or (II) therefore act as protective substances and restructuring agents.

In this manner, not only is it possible to provide a restructuring (repair) effect on damaged keratin fibers, but also a protective effect that counteracts damage to these materials before or during an exposure to noxious substances and may prevent or reduce it.

Besides these deleterious changes caused by exogenous noxae, the use according to the invention can produce advantageous effects also in the condition or changes in the structure of ke-ratin fibers brought about by physiological processes, for example in the case of hair embrittlement by age or fine hair that can be congenital or age-dependent (baby hair, old-age hair).

The use according to the invention consists of bringing the keratin-containing material, particularly keratin skin appendages and especially keratin fibers, most particularly hair, in contact with an agent containing an active ingredient in the form of a compound of formula (I) and/or (II), leaving it there after application or after an appropriate exposure time rinsing it off or out with an aqueous agent.

Preferably, the active ingredient of formula (I) and/or (II) is contained in the agent in an amount from 0.0005 to 30.0 weight percent, preferably from 0.001 to 20.0 weight percent, and most preferably from 0.001 to 10.0 weight percent, always based on the total amount of the agent.

The amount of active ingredient to be used depends strongly, for example, on the kind of effect it is intended to produce, on the hair and on the degree of hair damage. For example, it is possible to achieve a repairing and/or protective effect in the sense of the present invention by use of an amount of 0.001 weight percent of active ingredient of formula (I) in the betainic form. By use of very high concentrations of, for example, 20.0 weight percent of active ingredient in the form of the hydrochloride, a very intensive repairing and/or protective effect can be attained. Such an effect can be attained, in particular, on extremely damaged keratin-containing material, for example hair, or it can manifest itself in an advantageous manner before application of an unusually damaging treatment expected, for example, after repeated bleaching.

Hence, the present invention also comprises a method for repairing, strengthening, restructuring and protecting against mechanical and chemical damage keratin-containing material, characterized in that an agent containing a compound of formulas (I) and/or (II) 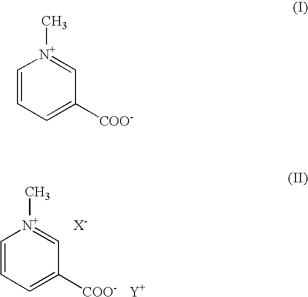
wherein X⁻ and Y⁻ denote any inorganic or organic, monovalent or polyvalent anion or cation, as already described hereinabove, also in the form of a plant extract, is brought in contact with the keratin-containing material for a period of 1 to 60 min at a temperature between 10° C. and 70° C. and then optionally rinsed out.

The agent described for the use according to the invention can be contained in all suitable formulations known in the cosmetic or pharmaceutical industry.

In particular, the agent can be in the form of an aqueous or aqueous-alcoholic solution, a gel, cream, emulsion or foam, it also being possible for the agent to be packaged in the form of a single-component preparation or in the form of a multicomponent preparation. In the case of a single-component preparation, the agent contains the active ingredient of formulas (I) and/or (II) together with suitable auxiliary or carrier materials known to those skilled in the art, for example thickeners, acids, fragrances, solvents, salts, wetting agent and/or organic and inorganic UV-filters, individually or in admixture.

If the agent is in the form of a multicomponent preparation, said agent can also consist of at least two different components kept spatially separated from one another up to the time of use. The first component can either contain as the active ingredient only the compounds of formulas (I) and/or (II) underlying the present invention, or the said component can contain the active ingredient together with an auxiliary substance (for example a thickener). A second or additional component contains only auxiliary and carrier materials.

It is also possible, however, that in a multicomponent preparation different components contain different active ingredients according to the present invention alone or in admixture with one another and with various auxiliary materials, and that the other components contain only auxiliary and carrier materials.

The invention comprises the use of a composition, characterized in that said composition is a single-component preparation or a multicomponent preparation. If the agent used is a multicomponent preparation, it comprises a first component containing the active ingredient of formula (I) and/or formula (II) with or without auxiliary substances and additives, and a second component containing the remaining constituents. Moreover, the agent used can be a multicomponent preparation with at least three different components wherein at least one of the components con-tains the active ingredient of formulas (I) and/or (II) and the other components contain the re-maining constituents.

Naturally, to prepare a ready-to-use agent, the spatially separated individual components of a mul-ticomponent preparation must be mixed with one another shortly before they are used according to the invention. In this regard, the reader is referred to WO 99/11222 which describes such a multicomponent system.

The agent of the invention can additionally contain carriers and auxiliary agents, for example solvents such as water, the lower aliphatic alcohols, for example ethanol, n-propanol and isopropanol, glycol ethers or glycols such as glycerol and particularly 1,2-propanediol; moreover dissolution promoters, wetting agents or emulsifiers from the classes of anionic, cationic, amphoteric or nonionic surface-active substances, such as fatty alcohol sulfates, ethoxylated fatty alcohol sulfates, alkyl sulfonates, alkylbenzene sulfonates, alkyltrimethylammonium salts, alkylbetaines, ethoxylated fatty alcohols, ethoxylated nonylphenols, fatty alkanolamides, ethoxylated fatty esters, furthermore thickeners such as the higher fatty alcohols, starch or cellulose derivatives; salts such as, for example, NaCl; buffering substances such as ammonium hydrogen carbonate; thiols, ketocarboxylic acids (oxocarboxylic acids), particularly α-ketocarboxylic acids, or the physiologically compatible salts thereof, UV absorbers, perfumes, dyes, conditioners, hair-swelling agents, preservatives, vaseline, paraffin oil and fatty acids, as well as hair-care agents such as cationic resins, lanolin derivatives, cholesterol, pantothenic acid and betaine; propellants such as, for example, propane, butane, dimethyl ether, N₂O, air and carbon dioxide and mixtures thereof.

The aforesaid constituents are used in amounts usually employed for such purposes, for example water in an amount from 0.1 to 95 wt. %, the wetting agents and emulsifiers at a total concentration from 0.2 to 30 weight percent, the alcohols in a total amount from 0.1 to 50 weight percent, the opacifiers, perfume oils, preservatives, and dyes in an amount from 0.01 to 5 weight percent each, the buffering substances in a total amount from 0.1 to 10 weight percent, the dissolution promoters, stabilizers, hair-conditioners and hair-care constituents in an amount from 0.1 to 5 weight percent each, whereas the thickeners and dissolution promoters can be contained in this agent in a total amount of 0.5 to 20 weight percent.

The pH of the agent is 2.0 to 14.0 and preferably 3.0 to 9.0. If necessary, the pH can be adjusted to the desired value by addition of an acid, for example an α-hydroxycarboxylic acid such as lactic acid, tartaric acid, citric acid or malic acid, phosphoric acid, acetic acid, glycolic acid, salicylic acid or gluconolactone, or of an alkalinizing agent such as ammonia, an alkanolamine, alkylamine, alkali metal hydroxide, ammonium hydroxide, alkali metal carbonate, ammonium carbonate or alkali metal phosphate.

In the treatment of keratin fibers, the agent containing the compounds of formulas (I) and/or (II) can remain there (for example in the hair) or it can be rinsed out after use. In the latter case, depending on the temperature (about 20 to 60° C. and preferably 30 to 50° C.), the time of exposure to the agent is 1 to 60 minutes, particularly 5 to 20 minutes, it being possible to accelerate the repairing action (restructuring) by supplying heat. The use of heat is therefore preferred. At the end of the exposure period, the hair can be rinsed with water and optionally washed with a shampoo.

The agent suitable according to the invention is preferably packaged in the form of a shampoo, rinse, treatment, foam, strengthening agent, hair gel, hair dye, hair tinting agent, permanent wave preparation, fixative, hair-smoothing agent or brillantine.

The agent can also be used as a pretreatment agent preceding chemical and/or physical treatment of keratin fibers, particularly before hair dyeing, hair tinting, hair bleaching or permanent hair deformation, for the purpose of preventing hair damage caused by these oxidative, reductive, acidic or alkaline treatments.

We were able to establish that the use according to the invention of an agent containing a compound of formula (I) and/or (II) of the present invention brings about a definite improvement in the structure of previously damaged keratin fibers, an improvement that can be proven not only by the afore-described test methods but also on the basis of a statistically highly significant increase in tear strength.

The results obtained by various measurements qualify the active ingredient of formula (I) and (II) unequivocally as a substance that can be used advantageously for repairing, strengthening and restructuring keratin-containing material and protecting it from mechanical and chemical damage.

### LEGEND FOR THE FIGURES

FIG. 1: Measurement of rubbing resistance of hair after tinting

1=natural hair

2=bleached hair

3=bleached hair plus tinting

4=as 3, but with 0.1% of 1-methylpyridinium-3-carboxylate

5=as 3, but with 1% of 1-methylpyridinium-3-carboxylate.

The measurements of rubbing resistance make it possible to draw conclusions concerning the resistance of the hair to mechanical stresses. The measuring principle consists of exposing individual hair fibers to repeated (small) mechanical stresses and measuring the number of repeating cycles needed to make the material break. A higher number of stressing cycles indicates a higher resistance of the hair to mechanical stresses. The evaluation of the hair resistance test results, which initially consists only of determining the number of stressing cycles to hair breakage for each individual hair, is carried out by Weibull's reliability analysis. According to Weibull, the breakage frequency (here: hair breakage) can be described by the following equation:
H=1−exp[−(x/Θ)]_{b}
linearized:
lnln[1/(1−H)]=b(ln x)−b(ln Θ)
H=breakage frequency (=cumulated hair breakage), normalized to 1 (1=100%)
X=lifetime variable (=cycles to hair breakage)
Θ=characteristic lifetime (number of cycles until 63.2% of the hairs are broken)
b=shape parameter, slope of the fitted straight lines.

For the Weibull analysis, therefore, that data must be plotted as lnln(1/1−H) against Inx. After linear regression, the axial segment indicates b(ln Θ) and the slope gives b. The characteristic lifetime E is then obtained from exp[−(axial segment/slope)]. The longer the characteristic lifetime, the higher is the resistance of the hair to breakage.

FIG. 2: Measurement of the rubbing resistance of hair after permanent wave treatment

1=natural hair

2=bleached hair

3=bleached hair plus permanent wave treatment

4=as 3, but with 0.5% of 1-methylpyridinium-3-carboxylate

FIG. 3: Measurement of dye abrasion

1=bleached hair plus tinting

2=as 1, but with 5% of 1-methylpyridinium-3-carboxylate

The following examples will explain the subject matter in greater detail. Unless otherwise indicated, the quantities of active ingredients, auxiliary agents and carriers indicated herein refer to the end product.

### EXAMPLE 1

### Determination of the Rubbing Resistance of Hair

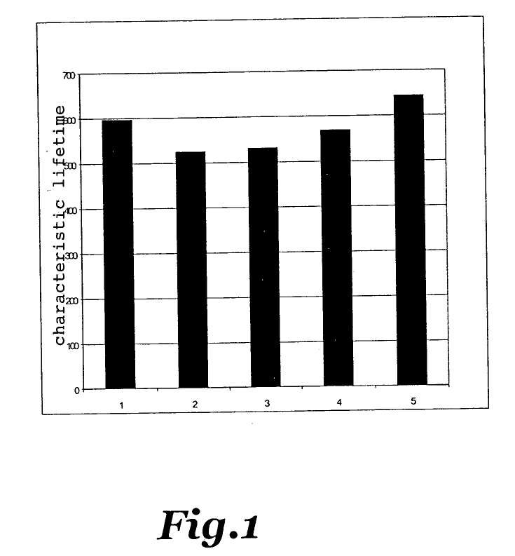

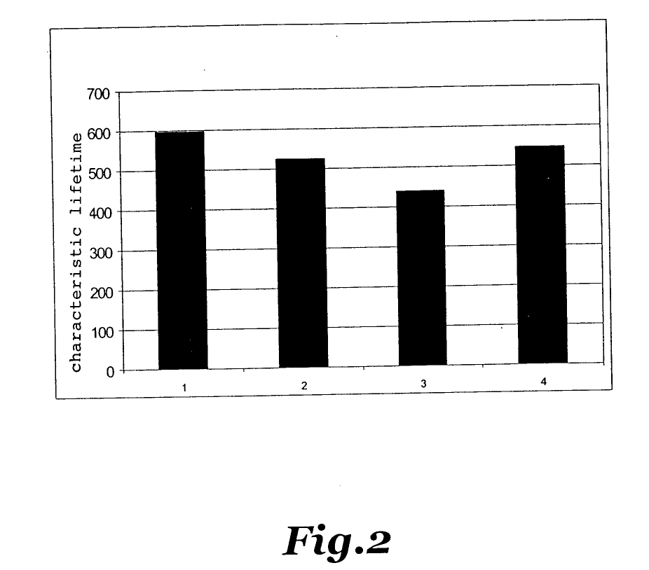

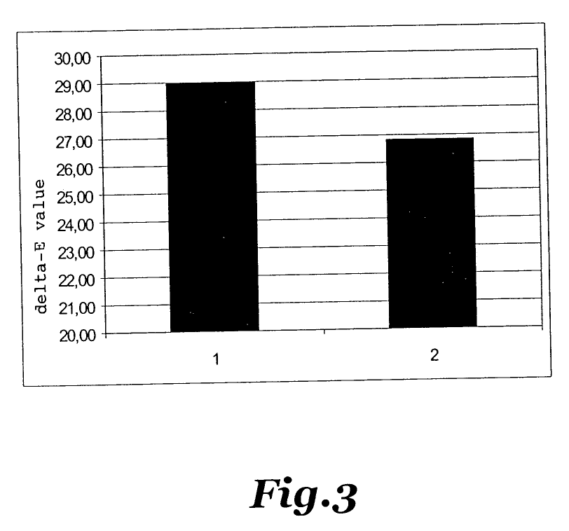

Another important parameter for determining the condition of the hair or the degree of damage to the hair is the determination of the rubbing resistance as a measure of the mechanical weakening of the hair (see legend for FIG. 1) [sic].

To this end, after previous standardized bleaching, strands of human hair (of Caucasian origin), were subjected to the strongly weakening conditions of a permanent wave procedure or a hair tinting by standardized methods. Then, the ends of 20 individual hairs per sample, namely of 10 individual hairs per strand, were fastened to holders, and the rubbing resistance was measured.

A pure hair tinting preparation (without 1-methylpyridinium-3-carboxylate) gave only a minimally longer characteristic lifetime of the hair than did the bleached but otherwise untreated hair. The addition of 0.1% of 1-methylpyridinium-3-carboxylate resulted in an increase, and the addition of 1% of 1-methyl-pyridinium-3-carboxylate to a further increase, in the characteristic lifetime of the hair. The values obtained for hair tinting at the 1% concentration of 1-methylpyridinium-3-carboxylate were even higher than those shown by the chemically untreated (unbleached, natural) hair. (FIG. 1).

Permanent wave processes reduce the characteristic lifetime of hair and thus its resistance to mechanical stresses. The addition of 0.5% of 1-methylpyridinium-3-carboxylate to permanent wave agents produced an increase in the characteristic lifetime of the hair. The value obtained was even higher than that for the bleached but not otherwise treated hair (FIG. 2).

TABLE 1
Rubbing Resistance Test Data After Hair Tinting

| Sample for Test in Hair Tinting Agent (FIG. 1) | Characteristic Lifetime |
|---|---|
| Natural | 596 |
| Bleached | 524 |
| Hair tinting agent, alone | 530 |
| Hair tinting agent + 0.1% of 1-methylpyridium-3- | 568 |
| carboxylate | |
| Hair tinting agent + 1% of 1-methylpyridium-3- | 643 |
| carboxytate | |

TABLE 2
Rubbing Resistance Test Data After Permanent Wave Treatment

| Sample for Test in Permanent Wave Agent | |
|---|---|
| (FIG. 2) | Characteristic Lifetime |
| Natural | 596 |
| Bleached | 524 |
| Standard permanent wave formulation | 439 |
| Permanent wave formulation with 0.5% of 1- | 546 |
| methylpyridium-3-carboxylate | |

### EXAMPLE 2

Determination of Triboelectric Properties of Hair

The determination of triboelectric properties, namely the determination of the electrostatic chargeability of hair, can also be used as a measure of the repairing or protective action on hair in the sense of the present invention. In this case, one makes use of the property that in tribological processes charges are created as a result of electron migration, which depending on the electromotive series produce a positive or negative charge or contact charge on the object involved.

In the antistatic measurement, the electrostatic charge in a volume of hair strand created by a plastic comb is determined by pattern analysis.

We used three strand volumes per sample, European hair, length: 22 cm, width: 2.5 cm, weight: 3.0 g.

To ensure average hair quality, the counting hair strands and the volume hair strands were first bleached for 30 min with bleaching powder (2.5 g/1 g of hair) and 9% Welloxon® (7.5 mL/1 g of hair), rinsed under running water (35° C.) for 2 minutes, then washed twice for 1 min with 0.5 mL of standard shampoo per 2 g of hair, rinsed 1 min and then dried at least overnight in a conditioning room at 20° C. and 65% relative humidity. The strands pretreated in this manner were then treated with the hair-treatment preparation containing 1-methylpyridinium-3-carboxylate (0.5%). After the hair had been exposed for 5 minutes, rinsed and dried, the hair strands were combed out 10 times with the coarse side of a commercial plastic comb before the measurement. Three untreated strands were used for control.

After being combed out, the hair strands treated in this manner had an obviously smaller volume than the untreated control hairs, which means that the treatments had reduced the electrostatic charge of the hair and thus also the hair volume. It was possible to demonstrate a statistically probable antistatic effect for the preparation containing 1% of 1-methylpyridinium-3-carboxylate as the hydrochloride.

### EXAMPLE 3

### Determination of Mechanical Abrasion of Artificial Dyes on Hair

By dye abrasion is meant the coloring of textiles by contact with moist or dried, colored hair. In practice, this property manifests itself in that parts of clothing become colored by brushing against dyed hair. The measurement of dye abrasion is intended to determine this effect.

The abrasion of hair dyes is a purely physical process. If the abrasion involves dry hair, the coloring of the objects is caused primarily by the mechanical detachment of dye particles from the hair and the transfer of said particles to the fabric. If abrasion involves moist hair, the effect of mechanical transfer is superposed by the bleeding of the dye from the hair strands and the resulting coloring of the textiles.

To measure the dye abrasion from dyed hair strands, a cotton tape was rubbed against a moist hair strand under constant pressure. The quantification of dye abrasion was performed by a known laboratory color measurement method (reflection measurement with a Minolta Spectrophotometer CM-508i). The dye distance ΔE (to the white textile tape) is a measure of the release of the dye from the hair. The higher the value of ΔE, the higher is the dye abrasion of the hair strands.

To this end, at least 30 g of a commercial hair-tinting formulation (Wella, Color Fresh Liquid) was applied to each of three dry combed strands. After an exposure time of 30 min at room temperature, the hair was rinsed under running water (35° C.) for 2 minutes and adjusted to a residual humidity of 50%.

A white cotton tape was then rubbed over each of the dyed strands under constant pressure in 300 cycles. The laboratory color determination of the dye abrasion effect was carried out the next day (FIG. 3).

TABLE 3
Abrasion Test Data

| | Content of 1-methylpyridinium- | Delta-E Value |
|---|---|---|
| | 3-carboxylate as Hydrochloride | (FIG. 3) |
| Sample 1 | — | 28.97 |
| Sample 2 | 5% | 26.83 |

### EXAMPLE 4 DETERMINATION OF THE TEAR STRENGTH OF HAIR

The determination of the tear strength of hair, which is an indicator of the structural integrity of the hair cortex and thus a measure of the degree of damage, was carried out by tensile-elongation measurements common for these purposes. From each hair strand we selected 20 individual hairs and determined the individual hair diameters with a computer-controlled laser micrometer. Then, by means of a tensile-elongation tester (MTT 160/600 Series Miniature Tensile Tester, Serial No. 600.95.05.001, supplied by DIA-STRON Ltd, England), the force needed to tear the individual hairs was measured.

From these individual tear strength measurements which because of the different hair diameters gave different values, the bundle tensile strength (BTS) was calculated by taking 0.08 mm for a hair diameter (average diameter). By including the hair density, finally, the conversion into the unit of bundle tensile strength (cN/tex) was performed. The higher the numerical value of the bundle tensile strength, the lower is the hair damage.

The measurement on hairs that had been treated with shampoo containing 1-methylpyridinium-3-carboxylate and with a conventional shampoo without 1-methylpyridinium-3-carboxylate gave the following results:

Damaged (blondized) hair was treated with the shampoo of Example 2 but without 1-methylpyridinium-3-carboxylate as hydrochloride. The quantity of 1-methylpyridinium-3-carboxylate was replaced with water. In this case, BTS=12.0±0.4 cN/tex (determined on 17 hairs from a shampoo-treated strand);

Damaged (blondized) hair treated with the shampoo as in the following Example 6 and with 2.0 wt. % of 1-methylpyridinium-3-carboxylate showed a BTS=12.64±0.4 cN/tex (determined on 20 hairs from a shampoo-treated strand).

The difference between the above-indicated average values is statistically highly significant (significance level by the t-test: 99.9%)

With 2.0 wt. % of 1-methylpyridinium-3-carboxylate, the bundle tensile strength was increased from 12.0 cN/tex to 12.64 cN/tex. The addition of 1-methylpyridinium-3-carboxylate thus brought about a definite strengthening of, or repair effect on, the hair

### EXAMPLES 5-7

Hair-Protection Shampoos

| | Amount in Wt. % | | |
|---|---|---|---|
| | 5 | 6 | 7 |
| Sodium lauryl ether sulfate | 40.0 | 40.0 | 40.0 |
| (25%) | | | |
| NaCl | 4.0 | 4.0 | 4.0 |
| 1-Methylpyridinium-3- | 0.2 | 2.0 | 5.0 |
| carboxylate hydrochloride | | | |
| Water | to 100 | to 100 | to 100 |

### EXAMPLE 8

Hair Shampoo for Intensive Protection

| | Amount in Wt. % |
|---|---|
| Sodium lauryl ether sulfate (25%) | 35.0 |
| NaCl | 2.0 |
| Triethanolamine | 4.0 |
| Phenoxyethanol | 0.08 |
| Methyldibromoglutaronitrile | 0.02 |
| Perfume oil | 0.1 |
| 1-Methylpyridinium-3-carboxylate | 20.0 |
| hydrochloride | |
| Water | to 100 |

### EXAMPLES 9-11

Hair-Protection Sprays

| | Amount in Wt. % | | |
|---|---|---|---|
| | 9 | 10 | 11 |
| Vinyl acetate/crotonic acid copolymer | 2.0 | 2.0 | 2.0 |
| 2-Amino-2-methyl-1-propanol | 0.160 | 0.160 | 0.160 |
| Ethanol (96%) | 37.840 | 37.840 | 37.840 |
| 1-Methylpyridinium-3-carboxylate | 0.001 | 0.005 | 0.010 |
| hydrochloride (betaine form) | | | |
| Perfume oil | 0.100 | 0.100 | 0.100 |
| Propane/butane (60:40) | to 100 | to 100 | to 100 |

### EXAMPLES 12-16

Cream Shampoos for Permanently Waved Hair

| | Amount in Wt. % | | | | |
|---|---|---|---|---|---|
| | 12 | 13 | 14 | 15 | 16 |
| Sodium lauryl sulfate | 10.0 | | | | |
| Sodium myristyl sulfate | | 10.0 | | | |
| Sodium cetyl sulfate | | | 10.0 | | |
| Sodium stearyl sulfate | | | | 10.0 | |
| Laureth-10 | | | | | 10.0 |
| Stearic acid | 9.0 | 9.0 | 9.0 | 9.0 | 9.0 |
| 1-Methylpyridinium-3- | 1.0 | 1.0 | 1.0 | 1.0 | 1.0 |
| carboxylate as acetate | | | | | |
| NaCl | 3.0 | 3.0 | 3.0 | 3.0 | 3.0 |
| Triethanolamine, pure | 4.0 | 4.0 | 4.0 | 4.0 | 4.0 |
| 1,2-Dibromo-2,4- | 0.1 | 0.1 | 0.1 | 0.1 | 0.1 |
| dicyanobutanine-2- | | | | | |
| phenoxyethanol | | | | | |
| Water | to 100 | to 100 | to 100 | to 100 | to 100 |

### EXAMPLES 17-21

Cream Shampoos for Permanently Waved Hair

| | Amount in Wt. % | | | | |
|---|---|---|---|---|---|
| | 17 | 18 | 19 | 20 | 21 |
| Sodium lauryl sulfate | 10.0 | | | | |
| Sodium myristyl sulfate | | 10.0 | | | |
| Sodium cetyl sulfate | | | 10.0 | | |
| Sodium stearyl sulfate | | | | 10.0 | |
| Laureth-10 | | | | | 10.0 |
| Stearic acid | 9.0 | 9.0 | 9.0 | 9.0 | 9.0 |
| 1-Methylpyridinium-3- | 3.0 | 3.0 | 3.0 | 3.0 | 3.0 |
| carboxylate as acetate | | | | | |
| NaCl | 3.0 | 3.0 | 3.0 | 3.0 | 3.0 |
| Triethanolamine, pure | 4.0 | 4.0 | 4.0 | 4.0 | 4.0 |
| 1,2-Dibromo-2,4- | 0.1 | 0.1 | 0.1 | 0.1 | 0.1 |
| dicyanobutanine-2- | | | | | |
| phenoxyethanol | | | | | |
| Water | to 100 | to 100 | to 100 | to 100 | to 100 |

### EXAMPLES 22-26

Cream Shampoos for Permanently Waved Hair

| | Amount in Wt. % | | | | |
|---|---|---|---|---|---|
| | 22 | 23 | 24 | 25 | 26 |
| Sodium lauryl sulfate | 10.0 | | | | |
| Sodium myristyl sulfate | | 10.0 | | | |
| Sodium cetyl sulfate | | | 10.0 | | |
| Sodium stearyl sulfate | | | | 10.0 | |
| Laureth-1 0 | | | | | 10.0 |
| Stearic acid | 9.0 | 9.0 | 9.0 | 9.0 | 9.0 |
| 1-Methylpyridinium-3- | 6.0 | 6.0 | 6.0 | 6.0 | 6.0 |
| carboxylate as acetate | | | | | |
| NaCl | 3.0 | 3.0 | 3.0 | 3.0 | 3.0 |
| Triethanolamine, pure | 4.0 | 4.0 | 4.0 | 4.0 | 4.0 |
| 1,2-Dibromo-2,4- | 0.1 | 0.1 | 0.1 | 0.1 | 0.1 |
| dicyanobutanine-2- | | | | | |
| phenoxyethanol | | | | | |
| Water | to 100 | to 100 | to 100 | to 100 | to 100 |

### EXAMPLES 27-31

Cream Shampoos for Permanently Waved Hair

| | Amount in Wt. % | | | | |
|---|---|---|---|---|---|
| | 27 | 28 | 29 | 30 | 31 |
| Sodium lauryl sulfate | 10.0 | | | | |
| Sodium myristyl sulfate | | 10.0 | | | |
| Sodium cetyl sulfate | | | 10.0 | | |
| Sodium stearyl sulfate | | | | 10.0 | |
| Laureth-10 | | | | | 10.0 |
| Stearic acid | 9.0 | 9.0 | 9.0 | 9.0 | 9.0 |
| 1-Methylpyridinium-3- | 10.0 | 10.0 | 10.0 | 10.0 | 10.0 |
| carboxylate as acetate | | | | | |
| NaCl | 3.0 | 3.0 | 3.0 | 3.0 | 3.0 |
| Triethanolamine, pure | 4.0 | 4.0 | 4.0 | 4.0 | 4.0 |
| 1,2-Dibromo-2,4- | 0.1 | 0.1 | 0.1 | 0.1 | 0.1 |
| dicyanobutanine-2- | | | | | |
| phenoxyethanol | | | | | |
| Water | to 100 | to 100 | to 100 | to 100 | to 100 |

### EXAMPLES 32-34

Intensive Hair-Restructuring Treatment

| | Amount in Wt. % | | |
|---|---|---|---|
| | 32 | 33 | 34 |
| Glycerol monostearate, neutral | 6.0 | 6.0 | 6.0 |
| Lanoline alkoxylate | 2.0 | 2.0 | 2.0 |
| 1-Methylpyridinium-3-carboxylate as citrate | 1.0 | 5.0 | 9.0 |
| Cetyl alcohol | 2.0 | 2.0 | 2.0 |
| Mixture of lanolin alcohol and paraffin oil | 1.0 | 1.0 | 1.0 |
| (1:9) | | | |
| Quaternium-52 | 1.5 | 1.5 | 1.5 |
| Hydroxyethylcellulose | 0.2 | 0.2 | 0.2 |
| Citric acid | 0.1 | 0.1 | 0.1 |
| Sorbic acid | 0.2 | 0.2 | 0.2 |
| Water | to 100 | to 100 | to 100 |

### EXAMPLES 35-36

Hair Treatment for Blondized Hair

| | Amount in Wt. % | |
|---|---|---|
| | 35 | 36 |
| Glycerol monostearate | 6.0 | 6.0 |
| Lanolin alkoxylate | 2.0 | 2.0 |
| Cetyl alcohol | 2.0 | 2.0 |
| Mixture of lanolin alcohol and paraffin oil (1:9) | 1.0 | 1.0 |
| Tris-(oligooxyethyl)alkyl ammonium phosphate | 1.5 | 1.5 |
| Hydroxyethylcellulose | 0.2 | 0.2 |
| Citric acid | 0.1 | 0.1 |
| Sorbic acid | 0.1 | 0.1 |
| Perfume oil | 0.1 | 0.1 |
| 1-Methylpyridinium-3-carboxylate hydrochloride | 0.5 | 1.5 |
| Water | to 100 | to 100 |

### EXAMPLE 37

Permanent Wave-Fixing Solution with Repair Function

| | Amount in Wt. % |
|---|---|
| Hydrogen peroxide | 4.6 |
| Citric acid | 0.2 |
| 1-Methylpyridinium-3-carboxylate hydrochloride | 0.5 |
| Perfume oil | 0.1 |
| Water | to 100 |

### EXAMPLE 38-39

Foam Conditioner for Protection Against Comb Damage

| | Amount in Wt. % | |
|---|---|---|
| | 38 | 39 |
| Polyquaternium-16 | 5.00 | 5.00 |
| VP/VA copolymer | 1.00 | 1.00 |
| Ceteareth-12 | 0.15 | 0.15 |
| Perfume oil | 0.10 | 0.10 |
| 1-Methylpyridinium-3-carboxylate hydrochloride | 1.00 | 2.00 |
| Propane/butane (60:40) | 10.00 | 10.00 |
| Water | to 100 | to 100 |

### EXAMPLES 40-41

Permanent Wave Formulation for Low Hair Damage

| | Amount in Wt. % | |
|---|---|---|
| | 40 | 41 |
| Ammonium thioglycolate (80%) | 9.5 | 9.5 |
| Ammonia (25%) | 1.6 | 1.6 |
| Ammonium carbonate | 4.5 | 4.5 |
| 1-Methylpyridinium-3-carboxylate (betaine form) | 0.5 | 3.0 |
| Perfume oil | 0.2 | 0.2 |
| Water | to 100 | to 100 |

### EXAMPLES 42-43

O/W Hair-Dressing Cream with Repair Function

Amount in Wt. %

| | 42 | 43 |
|---|---|---|
| Alkyl ether phosphate | 3.0 | 3.0 |
| Polyquaternium-22 | 1.2 | 1.2 |
| Paraffin oil | 17.0 | 17.0 |
| Perfume oil | 0.3 | 0.3 |
| Triethanolamine | 1.5 | 1.5 |
| Sodium formate | 0.5 | 0.5 |
| Trigonella foenum graecum extract¹ | 0.2 | 1.0 |
| Water | to 100 | to 100 |

¹Aqueous extract, deproteinized, defatted and concentrated . . . Active content, calculated as 1-methylpyridinium-3-carboxylate in betaine form: 10%

One kilogram of ground carob seed was heated under reflux with 5 liters of ethanol/water (60% ethanol) for 2 hours. The mixture was then filtered, and the filter residue was extracted twice with three liters of 60% ethanol. From the combined filtrates, the ethanol part was distilled off. The remaining aqueous phase was acidified with formic acid to pH 2-3 and, to remove the fat, shaken three times with one liter-portions of ethyl acetate. The aqueous phase was then concen-trated to about 10 g, and the exact content of 1-methylpyridinium-3-carboxylate was determined by HPLC. The active ingredient content was then adjusted to 10% by further concentration or by addition of water. This gave from 8 to 15 g of extract. The remaining residue of formic acid ex-erted a preserving action at the same time.

### EXAMPLES 44-45

Hair-Firming Agent for Long Hair

| | Amount in Wt. % | |
|---|---|---|
| | 44 | 45 |
| Luviskol VA 55E | 6.0 | 6.0 |
| Perfume oil | 0.3 | 0.3 |
| Ethanol (96%) | 40.0 | 40.0 |
| 1-Methylpyridinium-3-carboxylate hydrochloride | 0.2 | 2.0 |
| Water | to 100 | to 100 |

### EXAMPLES 46-47

Tinting Foam for Low Dye Abrasion

| | Amount in Wt. % | |
|---|---|---|
| | 46 | 47 |
| Ethanol (96%) | 10.0 | 10.0 |
| Polyquaternium-11 | 7.5 | 7.5 |
| Basic Violet 2 | 0.2 | 0.2 |
| Cyclomethicone | 0.2 | 0.2 |
| Perfume oil | 0.3 | 0.3 |
| 1-Methylpyridinium-3-carboxylate hydrochloride | 0.5 | 2.5 |
| Water | to 100 | to 100 |
| Propane/butane (60:40) | 10.0 | 10.0 |

### EXAMPLES 48-49

Hair-Coloring Cream for Low Dye Abrasion

| | Amount in Wt. % | |
|---|---|---|
| | 48 | 49 |
| Stearyl alcohol | 8.00 | 8.00 |
| Paraffin oil | 13.00 | 13.00 |
| Wool grease | 6.00 | 6.00 |
| Perfume | 0.30 | 0.30 |
| p-Toluenediamine | 0.70 | 0.70 |
| Resorcinol | 0.05 | 0.05 |
| Aminophenol | 0.06 | 0.06 |
| EDTA | 0.20 | 0.20 |
| Ammonia (25%) | 2.00 | 2.00 |
| Sodium sulfite | 1.00 | 1.00 |
| 1-Methylpyridinium-3-carboxylate hydrochloride | 1.00 | 5.00 |
| Water | to 100 | to 100 |

### EXAMPLES 50-51

Hair Tonic for Frequent Combing

Amount in Wt. %

| | 50 | 51 |
|---|---|---|
| Ethanol (96%) | 60.00 | 60.00 |
| Perfume oil | 0.15 | 0.15 |
| Panthenol | 0.20 | 0.20 |
| Luviskol K 30 | 0.05 | 0.05 |
| Salicylic acid | 0.10 | 0.10 |
| Menthol | 0.02 | 0.02 |
| Camphor | 0.01 | 0.01 |
| Allantoin | 0.10 | 0.10 |
| Coffea arabica¹ extract | 10.00 | 20.00 |
| Water | to 100 | to 100 |

¹Aqueous extract from ground, unroasted coffee beans, deprotinized, defatted and concentrated. Active content, calculated as 1-methylpyridinium-3-carboxylate in the betaine form: 10%

One kilogram of ground, unroasted coffee beans was heated under reflux with 5 liters of ethanol/water (60% ethanol) for 2 hours. The mixture was then filtered, and the filter residue was extracted twice with three liters of 60% ethanol. From the combined filtrates, the ethanol part was distilled off. The remaining aqueous phase was acidified to pH 2-3 with formic acid and, to remove the fat, shaken three times with one liter-portions of ethyl acetate. The aqueous phase was then concentrated to about 100 g, and the exact content of 1-methylpyridinium-3-carboxylate was determined by HPLC. The active ingredient content was then adjusted to 10% by further concentration or by addition of water. This gave from 60 to 150 g of extract. The remaining residue of formic acid exerted a preserving action at the same time.

1. Use of a compound of formula (I) and/or (II) 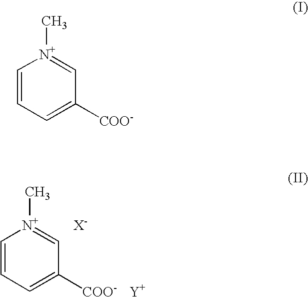
and of the salts thereof, wherein X⁻ and Y⁺ stand for any inorganic or organic, monovalent or polyvalent physiologically unobjectionable anion or cation, in an agent for repairing, strengthening and restructuring keratin-containing material and protecting such material against mechanical and chemical damage.

2. Use as defined in claim 1, characterized in that the compounds of formulas (I) and/or (II) are of synthetic or natural origin.

3. Use as defined in claim 1, characterized in that the compounds of formulas (I) and/or (II) are present as constituents of a natural plant extract.

4. Use as defined in claim 1, characterized in that the active ingredient is in its pure, betainic form or is present as a salt of the compound of formula (II).

5. Use as defined in claim 1, characterized in that X⁻ is selected from the group consisting of formate, tartrate, oxalate, aspartate, glutamate, acetate, citrate or of an inorganic group such as chloride, bromide, iodide, sulfate, hydrogen sulfate, phosphate, monohydrogen or dihydrogen phosphate, hydroxide, carbonate and nitrate and that Y⁺ is selected from the group consisting of protons, alkali metals, preferably lithium, sodium and potassium, alkaline earth metals, preferably calcium and magnesium, subgroup metals, preferably aluminum, iron, zinc, copper, manganese and silver, ammonium groups or primary, secondary, tertiary or quaternary amines, hydrazides or hydroxylammonium groups.

6. Use as defined in claim 1 on weakened and/or damaged keratin-containing material.

7. Use as defined in claim 1, characterized in that before, during or after expo-sure of the keratin-containing material to chemical and/or physical noxae, the agent is brought in contact with the keratin-containing material.

8. Use as defined in claim 1 in a pretreatment agent before a chemical and/or physical treatment of keratin-containing material.

9. Use as defined in claim 1 for protecting or reducing damage the inner structure of keratin-containing material or for repairing (restructuring) said keratin-containing material.

10. Use as defined in claim 1, characterized in that the chemical treatment comprises dyeing, tinting, bleaching or permanent deformation.

11. Use as defined in claim 1, characterized in that the keratin-containing material consists of skin appendages.

12. Use as defined in claim 11, characterized in that the skin appendages are keratin fibers.

13. Use as defined in claim 1 for cosmetic treatment of sensitive, brittle and/or fine keratin fibers.

14. Use as defined in claim 12, characterized in that the keratin fibers are human hair.

15. Use as defined in claim 1, characterized in that the compounds of formulas (I) and/or (II) are contained in the agent in an amount from 0.001 to 30.0 weight percent, based on the total amount.

16. Use as defined in claim 1, characterized in that the compounds of formulas (I) and (II) are contained in the agent in an amount from 0.05 to 10.0 weight percent, based on the total amount.

17. Use as defined in claim 1, characterized in that the agent is in the form of an aqueous or aqueous-alcoholic solution, emulsion, foam, cream or gel.

18. Method for repairing, strengthening and restructuring keratin-containing material and for protecting such material from mechanical and chemical damage, characterized in that an agent containing a compound of formula (I) and/or II) 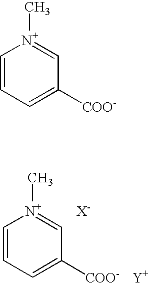
and the salts thereof, wherein X⁻ and Y⁺ stand for any inorganic or organic monovalent or polyva-lent physiologically unobjectionable anion and cation, or in the form of a plant extract, is brought in contact with the keratin-containing material at a temperature between 10 and 70 θC for a period of 1 to 60 minutes and is then optionally rinsed out.
